(19) United States
(12) Patent Application Publication
(10) Pub. No.: US 2024/0157010 A1
(43) Pub. Date: May 16, 2024

(54) DEVICE AND CONTROL METHOD FOR DISINFECTING OBJECTS

(71) Applicant: PRATRIVERO S.P.A., VALDILANA (BI) (IT)

(72) Inventor: Sergio Barberis Canonico, VALDILANA (BI) (IT)

(73) Assignee: PRATRIVERO S.P.A., VALDILANA (BI) (IT)

(21) Appl. No.: 18/549,707

(22) PCT Filed: Mar. 15, 2022
(86) PCT No.: PCT/IB2022/052313

§ 371 Date: Sep. 8, 2023

(30) Foreign Application Priority Data
Mar. 15, 2021 (IT) 102021000006113

(51) Int. Cl. A61L 2/24; A61L 2/10

(52) U.S. Cl. CPC A61L 2/24; A61L 2/10; A61L 2202/11; A61L 2202/122; A61L 2202/14

(57) ABSTRACT

A device for disinfecting objects comprising: a box-shaped body, at least one cold cathode lamp, mounted internally the box-shaped body and configured to irradiate the object to be disinfected with an ultraviolet radiation, one or more detection devices configured to detect environmental conditions, a control system configured to determine the activation time required for the at least one lamp to reach a nominal ultraviolet radiation output. The control system being further configured to control the power supply of the at least one lamp.

### CROSS-REFERENCE TO RELATED APPLICATIONS

This patent application claims priority from Italian patent application no. 102021000006113 filed on Mar. 15, 2021, the entire disclosure of which is incorporated herein by reference.

### TECHNICAL FIELD

The present invention relates to a device and a method for disinfecting objects.

### STATE OF THE ART

The germicidal capacity of the electromagnetic outputs (rays) within the ultraviolet spectrum (UV) is commonly known and is applied to devices for disinfecting objects, tools, air, water. In such devices, the output of the UV rays normally occurs by means of discharge lamps or, more recently, LEDs. Output frequencies being equal, the amount of time in which it is possible to obtain a satisfactory disinfection closely depends on the dose of UV rays that hits the object to be disinfected, i.e. on the closeness thereof to one or more outputting elements and on the power of the latter.

UV emitting lamps, especially for the high powers, not yet reachable by LED lamps, are generally discharge lamps containing mercury vapours, which can substantially be grouped into two categories: hot cathode lamps and cold cathode lamps.

Hot cathode lamps are commonly the most widespread and have various characteristics to their advantage among which low cost and relatively high efficiency. However, their life cycle is strongly affected by the number of previous switching on operations and require a relatively long amount of time (minutes or dozens of minutes) between the moment of the switching on and the reaching of the nominal UV output required for carrying out an accurate disinfection.

On the other hand, the cold cathode lamps obviate the deterioration caused by the number of switching on, but they too require a high amount of time from the switching on to the maximum (or nominal) output of UV rays.

Both types of the abovementioned lamps convert into UV radiation only a certain percentage of the energy used (usually approximately 30%); the remaining part is for the most part converted into heat, which requires to be dissipated for not prejudicing the life of the lamp.

Therefore, the use of UV lamps is required if it is necessary to reach high powers. In fact, these lamps prove to be highly performing for all the applications in which said lamps remain switched on for a long amount of time, where the initially required time for reaching the nominal output is not relevant, where the heat produced can be suitably dissipated.

In a period when disinfecting objects has become increasingly important due to the pandemic spreading of pathogens, the need is thus felt to develop a device that can be used for rapidly disinfecting common objects, so as to easily allow the exchange thereof between a plurality of individuals without running into risks of infection.

### SUBJECT OF THE INVENTION

The object of the present invention is to provide a device and a relative control method which allow overcoming, at least partially, the drawbacks of the prior art and which are, at the same time, easy and cost-effective to embody.

In accordance with the present invention, a device for disinfecting objects and a relative control method are provided according to what indicated in the following independent claims and, preferably, in any one of the claims directly or indirectly dependent on the independent claims.

The claims describe preferred embodiments of the present invention forming integral part of the present description.

### BRIEF DESCRIPTION OF THE DRAWINGS

For a better understanding of the present invention, a preferred embodiment is described in the following, simply by way of non-limiting example and with reference to the accompanying drawings, wherein:

FIG. 1 is a schematic and perspective view of a first embodiment of a disinfecting device according to the present invention;

FIG. 2 is a schematic and front view of the connections between some elements of the disinfecting device according to FIG. 1;

FIG. 3 is a flow diagram which illustrates the possible steps of a control method for controlling a disinfecting device in accordance with the present invention.

### DETAILED DESCRIPTION OF THE INVENTION

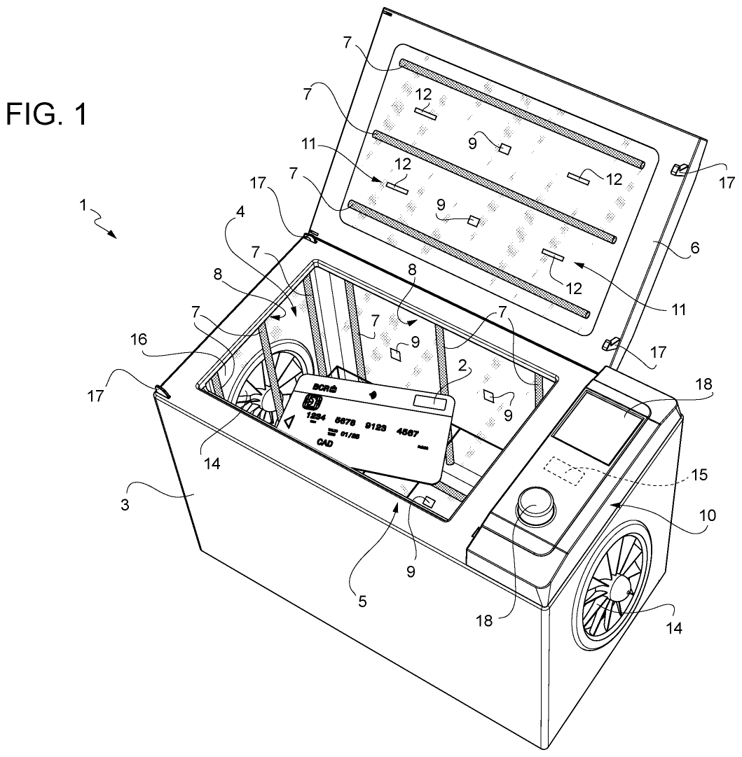

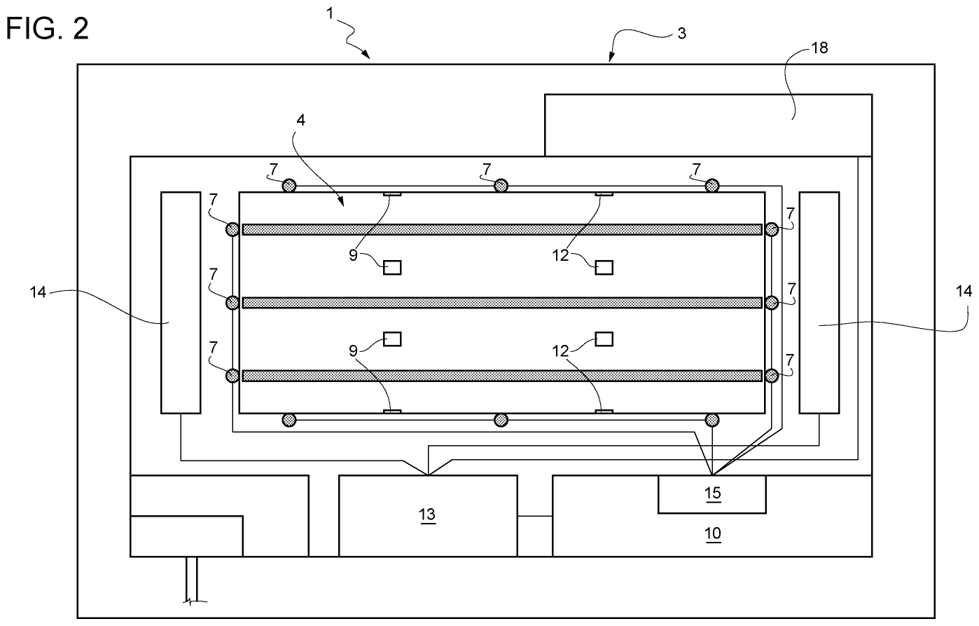

In FIGS. 1 and 2, reference numeral 1 indicates, as a whole, a device for disinfecting objects 2. In particular, in FIG. 1 the object 2 to be disinfected is a credit card. However, the device 1 can be used for disinfecting any type of object 2 that can be inserted therein.

The device 1 comprises a box-shaped body 3, which determines therein a disinfection chamber 4 configured to accommodate the object 2 to be disinfected.

Furthermore, the box-shaped body 3 is openable on at least one portion 5 thereof for allowing inserting the object 2 from the outside of the device 1 to the inside of the disinfection chamber 4.

In the non-limiting embodiment of FIG. 1, the box-shaped body has a parallelepiped shape, in which the top face corresponds to the portion 5 and is provided with an openable and hinged door 6. In other non-limiting embodiments, the box-shaped body has different shapes and/or the door 6 is made so as to open frontally or laterally. Preferably, the door 6 is openable towards the outside, so as not to interfere, during the closing, with the objects 2 inside the chamber 4.

Advantageously, the device 1 further comprises at least one cold cathode lamp 7, mounted internally the box-shaped body 3 and configured to irradiate the object 2 to be disinfected with an ultraviolet radiation (i.e. having a wavelength between 400 and 100 nanometres). In particular, the lamp 7 is configured to output UV-C (between 280 and 100 nanometres) or UV-C+ozone radiations.

Preferably, the device 1 comprises a plurality of lamps 7 arranged in a uniform manner on an inner surface of the box-shaped body 3 (more precisely on a plurality of faces 8 thereof). In particular, in the non-limiting embodiment of FIG. 1, three lamps 7 are present for each face 8 of the inner surface of the box-shaped body 3. Specifically, the lamps 7 have an elongated shape and are arranged, on a same face 8, parallel to one another, in particular they are equally distributed on the face 8. In such manner, it is possible to irradiate the object 2 from multiple angles.

In some non-limiting cases, the object 2 can be arranged on a support (preferably transparent to the UV-C radiation or which anyway allows the passing thereof) which substantially places it in the centre of the disinfection chamber 4, so that it can be equally irradiated by the lamps 7 arranged around it when it is inside the device 1.

Advantageously, the device 1 further comprises one or more detection devices 9 configured to detect environmental conditions inside the disinfection chamber 4. In particular, il device 1 comprises a plurality of detection devices 9, so as to be able to detect the environmental conditions in the proximity of each lamp 7.

According to some preferred embodiments, the environmental conditions comprise the temperature in the proximity of each lamp 7 (or the temperature of the lamp 7). Alternatively or additionally, the environmental conditions comprise the humidity and/or the radiation. Temperature, humidity and radiation sensors are known per se and therefore will not be described in further detail.

In the non-limiting embodiment of FIG. 1, the detection devices 9 are arranged symmetrically between the lamps 7.

Advantageously, the device 1 further comprises a control system 10 configured to determine, based on at least the detected environmental conditions and the inactivity time of the device 1 (in particular of each lamp 7), an activation time required for the (or for each) lamp 7 for reaching (from the current condition) a nominal ultraviolet radiation output. For example, the nominal radiation output is the maximum output of the respective lamp 7. In particular, the nominal radiation output is an ultraviolet output which allows disinfecting the object 2 by at least 99.9% (the so-called “3-log”), preferably at least 99.99% (the so-called “4-log”) of the pathogens present on the object. More in particular, such disinfection occurs keeping into account the inactivity time of the lamp 7, the environmental conditions and the current working point of the lamp 7 within its life cycle (whose graphs are generally provided by the producers of the lamps 7, or can be derived experimentally).

Advantageously, the control system 10 is further configured to control the power supply (or driving) current of each lamp 7, so as to adjust the ultraviolet output and, indirectly, the temperature thereof.

In particular, the adjustment of the power supply current is used for adjusting the UV-C or UV-C+ozone dose output by the lamp 7 and/or the temperature of the lamps 7 or of the surrounding environment thereof.

In the non-limiting embodiment of FIG. 2, the control system 10 is singularly connected to each lamp 7. In other non-limiting cases not illustrated, the control system 10 is series-connected to the lamps 7.

Preferably, the device 1 also comprises an auxiliary lighting system 11 arranged at the lamps 7 and configured to switch on so as to reduce the activation time. In particular, the system 11 is a LED system, comprising a plurality of luminous elements 12 (LEDs) which switch on (continuously or intermittently) for quickening the triggering and the switching on of the lamps 7. In such manner, the activation time of the lamps 7 in environments devoid of light is eased by the simultaneous switching on of one or more LED luminous elements 12 operating in the visible spectrum placed in the proximity of the lamps 7. In fact, the arc triggering time of a cold cathode lamp also depends on the ambient light in the spectrum of the visible; therefore, by suitably lighting the lamps 7 for the instant in which the arc is triggered, such operation is quickened.

Advantageously but not necessarily, and as illustrated in the non-limiting embodiment of FIG. 1, the device 1 further comprises an active thermal stabilization system 13 arranged at each lamp and configured to dynamically adjust the surrounding temperature thereof. For example, the thermal system 13 comprises one or more Peltier cells or uses the thermal power dissipated by the lamps for adjusting the temperature in the surroundings of the same at a value considered suitable.

Preferably, the device 1 further comprises at least one dissipation element 14, configured to dissipate at least part of the heat generated by the lamps 7 and convey it to the outside of the device 1. For example, the dissipation element 14 is an electrically operated fan. In particular, the device 1 comprises at least two fans arranged on two faces opposite the box-shaped body 3. In such manner, it is possible to generate a continuous and diffused air flow inside the device 1.

Preferably, the control system 10 is configured to dynamically adjust the temperature at the lamps 7 varying the generated heat dissipation of the element 14 and/or the operation of the system 13.

Advantageously but not necessarily, the control system 10 is configured to maintain, in the absence of an object 2 to be disinfected inside the disinfection chamber 4, at least one lamp 7 partially powered in a standby configuration. The term “standby configuration” means a pre-heating configuration, in which the lamp 7 is kept partially powered (switched on), at (or in order to reach) a certain temperature and with the respective current arc at a minimum predetermined steady state (uninterrupted), in order to minimise the activation (or reactivation) time.

In some preferred but non-limiting cases, the device 1 further comprises an analogue (instead of pulse train) dimming element 15 for controlling the power supply current of each lamp 7. In such manner it is possible to optimise the duration and the yield of the lamp 7.

However, the present device 1 works correctly also in the event of pulse train dimming.

Advantageously but not necessarily, the device 1 comprises reflecting elements 16 arranged inside the disinfection chamber 4 (for example, as illustrated in FIG. 1, on the faces 8 of the disinfection chamber 4) so as to convey the ultraviolet radiation towards the object 2 to be disinfected.

According to some non-limiting embodiments (as illustrated in FIG. 1), the device 1 comprises safety means 17 configured to interrupt (or limit) the power supply of the lamps 7 in the event of opening of the portion 5 (i.e. of the door 6) of the box-shaped body 3.

For example, the safety means 17 can be mechanical, optical or magnetic. In use, in the event a user opens the device 1 during the disinfection (and thus with the lamps 7 in the ultraviolet output step), the safety means 17 promptly intervene cutting the power supply of the lamps and preventing any risks for the user.

Advantageously but not necessarily, the control system 10 is further configured to store each radiation cycle of each lamp 7 and consequently to estimate the decay of the lamps 7 based on the data normally provided by the supplier of the lamps or determined empirically through specific laboratory tests. In particular, the decay of the lamps 7 is used for determining the abovementioned current working point within the life cycle of the lamps 7.

In some advantageous and non-limiting cases, the detection devices 9 comprise sensors for measuring the UV-C radiation, so as to be able to dynamically detect over time and with precision the dose of UV rays output by the lamps 7, the radiation capacity of the lamps 7 and/or the decay of the same.

According to some preferred but non-limiting embodiments, the detection devices 9 comprise means configured to detect the presence, the geometry and/or the dimension of the object 2 to be irradiated in order to optimise the amount of time and the modes of exposure. In particular, such means comprise optical devices configured to detect shapes, bulks and/or distances.

Preferably, the device 1 comprises an interface element 18, for example a touch screen, or a control push-button panel, configured to allow the user to select different types of disinfection (adjusting, by means of the control system 10, the dose of ultraviolet radiations output). In particular, the interface device 18 is further configured to emit a sonorous and/or visual notice upon the completion of the disinfection.

In accordance with a second aspect of the present invention, a control method for controlling a device for disinfecting objects is provided, in particular of the type described above.

The method comprises at least the steps of: detecting the environmental conditions inside the disinfection chamber 4 determined by the box-shaped body 3; determining (i.e. calculating) the inactivity time of the device 1 (i.e. of the lamps 7, or of each lamp 7); and elaborating (calculating), based on the environmental conditions and the inactivity time, the activation time necessary for the cold cathode lamps 7 (or for each cold cathode lamp 7) to reach the nominal ultraviolet radiation output. In particular, the method comprises the further step of cyclically verifying that the elaborated activation time is below a threshold value (for example equal to thirty seconds or less—depending on the intended degree of disinfection of the object 2 to be reached).

The method further comprises the step of controlling (adjusting) the power supply of the at least one lamp 7 so as to keep it, even in the absence of an object 2 to be disinfected inside the device 1 (i.e. the disinfection chamber 4), at least partially powered in the standby configuration, in order to keep the activation time below the predefined threshold value.

Advantageously but not necessarily, the method comprises at least the further step of disinfecting the object 2 arranged inside the device 1 (i.e. the disinfection chamber 4) for a time in the order of tens of seconds (in particular less than 80 seconds, preferably less than 60 seconds, more in particular less than 40 seconds) by removing 99.9% of the pathogens. In particular, during the disinfection step, the lamps 7 are switched on and powered for providing the object 2 with a sufficient quantity of UV rays so as to allow a thorough disinfection (at least “3-log”, preferably “4-log”). More in particular, the disinfection of the object 2 is carried out within thirty seconds.

According to some preferred non-limiting embodiments, the method provides for switching on at least one LED auxiliary lighting system 11 arranged at the at least one lamp 7 so as to reduce the activation time in accordance with what said in the foregoing.

Preferably, the adjustment of the power supply (driving) current by the control system 10, occurs in a continuous (analogue) manner. In particular, by means of such adjustment, the dose of UV rays output and/or the temperature of the lamps 7 or of the surrounding environment thereof is controlled. Therefore, the adjustment of the current (and/or of the switching on and switching off cycles of the lamps) allows being able to calibrate with precision the (germicidal) dose of UV rays during the disinfection step.

According to the non-limiting embodiment of the accompanying figures, the method further comprises a (continuous) step of controlling the temperature (in the proximity) of the lamps 7. During such step the control system 10 dynamically adjusts the temperature at the lamps 7 varying the generated heat dissipation of the element 14 and/or the operation of the system 13.

Preferably, the method further provides for the control system 10 to store each radiation cycle of each lamp 7 and consequently to estimate the decay of the lamps 7 based on the data normally provided by the supplier of the lamps or determined empirically on similar lamps. In particular, the decay of the lamps 7 is used by the control system 10 for determining the abovementioned current working point within the life cycle of the lamps 7.

According to some non-limiting embodiments, during the detection step, the devices 9 further detect, dynamically over time and with precision, the dose of UV rays output by the lamps 7, the radiation capacity of the lamps 7 and/or the decay of the same.

Advantageously but not necessarily, the method further provides for detecting the presence, the geometry and/or the dimension of the object 2 to be irradiated in order to optimise the amount of time and the modes of exposure.

Advantageously but not necessarily, the method is carried out by a device 1 according to what described up to here.

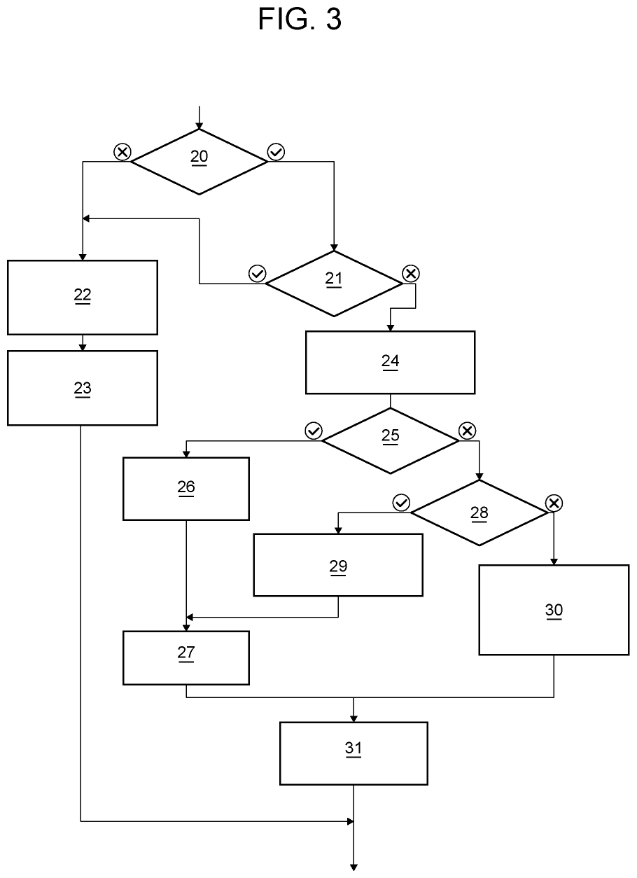

In the following, reference will be made to the non-limiting embodiment of FIG. 3, which is a possible non-limiting embodiment of the control method described above. In particular, in the flow diagram of FIG. 3 the convention was used according to which the rectangular blocks indicate a general instruction and the rhomboidal blocks, placed at a branch, are choice blocks, containing a logic condition which determines the direction in which the flow will go. In particular, at the choice blocks, the flow of the diagram rebranches in the direction marked by the check symbol “✓” if the logic condition is met, otherwise, in the event such condition is not met, the flow rebranches in the direction marked by the symbol “X”.

In use, as illustrated in the flow diagram of FIG. 3, the method firstly provides for verifying (block 20) the possibility of controlling and powering the lamps 7. In particular, such possibility depends on the decay state of the lamps and on the safety conditions (for example, if the device 1 is open, it will not be possible to power the lamps).

In the event the adjustment is possible, the method provides for verifying if a switch off command of the device 1 is present (block 21). In the event such command is present, the control system 10 switches off the lamps 7 (block 22) and therefore the dose of UV rays (i.e. the dose from which the disinfection derives) output inside the disinfection chamber 4 tends to zero (block 23).

Otherwise, if the adjustment is possible and there is no switch off command, the control system 10 proceeds in elaborating (block 24) the activation time (of each lamp or of each set of lamps) depending on the current temperature of the lamps 7 or the surrounding environment thereof, depending on the inactivity time of the lamps 7 and the total working time of the lamps from the beginning of their lives.

Subsequent to the calculation of the activation time, it is verified (block 25) that the latter falls within (i.e. is below) the predefined threshold value. In the event such value exceeds the abovementioned threshold value (i.e. the time required for reaching the nominal UV output is above what desired), the control system 10 determines the start of a pre-heating step, during which it drives the lamps 7 for reaching as soon as possible a provided temperature for the standby configuration (block 26) and consequently for lowering the value of the activation time below the predefined threshold. Subsequent to this step, the dose (quantity) of UV rays output by the lamps 7 is anyway calculated (block 27). In particular, by detecting such dose it is possible to improve the control of the lamps (so as to keep the outputs below a value which could be harmful for a user who inserts the object 2 to be disinfected inside the device 1).

Whereas, when the value of the calculated activation time falls within the predefined threshold value (block 28), the control system 10 verifies if a disinfection command has been given. If it has, the lamps 7, the system 13 and the elements 14 are consequently activated so as to proceed with the step of disinfecting the object 2 according to the degree and the amount of time demanded by the user by means of the interface device 18 (or predefined, in the event of a mono-mode device 1). In such case, the subsequent block is block 27, detecting (or calculating) the dose of UV rays output by the lamps 7. On the other hand, in the absence of a disinfection command (block 30) the lamps 7 are kept in the standby configuration, ready to carry out their disinfecting function in less time possible.

Subsequent to the abovementioned steps, the method provides for verifying the operating state of the lamps 7 (or of each lamp 7) within block 31, so as to update the total working time of the lamps (or of each lamp) 7, i.e. the current working point of the lamp 7 within the life cycle thereof.

In the non-limiting embodiment of FIG. 3, the abovementioned flow diagram repeats cyclically.

Although the above-described invention particularly refers to some very specific example embodiments, it is not limited to such example embodiments, falling within its scope all the variations, modifications or simplifications covered by the appended claims, such as for example different safety means, a different shape of the box-shaped body, a different flow of the steps of the method, a different arrangement of the lamps and/or of the reflecting elements, etc.

The device and the method described above entail numerous advantages.

Firstly, they allow, using a moderate-sized object which does not generate excessive heat, being able to place inside money and/or credit cards (or any other object), subjecting it all to disinfection with known doses and quickly.

Furthermore, both the excessive consumption that there would be in perpetually keeping the ultraviolet lamps in steady state, and the risk generated by the exposure of the users during the insertion and the collection of the object to be disinfected/disinfected are synergically reduced.

Additionally, the device and the method allow sensibly decreasing the time necessary for arriving at the nominal 5 output of the cold cathode lamps and therefore the disinfection time.

Finally, the device 1 allows having an intermittent working without excessively penalising the life of the lamps 7.

1. A device (1) for disinfecting objects (2) comprising:
a box-shaped body (3), which determines inside thereof a disinfection chamber (4) and is openable on at least one portion (5) thereof to allow inserting an object (2) to be disinfected inside the disinfection chamber (4);
at least one cold cathode lamp (7), mounted internally the box-shaped body (3) and configured to irradiate the object (2) to be disinfected with an ultraviolet radiation;
one or more detection devices (9) configured to detect environmental conditions inside the disinfection chamber (4);
a control system (10) configured to determine, based on at least the detected environmental conditions and the inactivity time of the device (1), an activation time required for the at least one lamp (7) to reach a nominal ultraviolet radiation output; the control system (10) being further configured to control the power supply of the at least one lamp (7).

2. The device (1) according to claim 1, comprising at least one auxiliary lighting system (11) arranged at the at least one lamp (7) and configured to switch on in order to reduce the activation time.

3. The device (1) according to claim 1, comprising at least one active thermal stabilisation system (13) arranged at the at least one lamp (7) or centralised.

4. The device (1) according to claim 1, wherein the control system (10) is configured to maintain, in the absence of an object (2) to be disinfected inside the disinfection chamber (4), the at least one lamp (7) partially powered in a standby configuration.

5. The device (1) according to claim 1, wherein the control system (10) comprises an analogue or digital dimming element (15) for controlling the power supply of the at least one lamp (7).

6. The device (1) according to claim 1 and comprising reflecting elements arranged inside the disinfection chamber (4) so as to convey the ultraviolet radiation towards the object (2) to be disinfected.

7. The device (1) according to claim 1 and comprising safety means (17) configured to interrupt the power supply of the at least one lamp (7) in the event of opening of the portion (5) of the box-shaped body (3).

8. A method for controlling a device (1) for disinfecting objects (2) comprising at least one box-shaped body (3) inside which the at least one cold cathode lamp (7) is housed; the method comprising the steps of:
detecting environmental conditions inside a disinfection chamber (4) determined by the box-shaped body (3);
determining the inactivity time of the device (1);
elaborating, on the basis of the environmental conditions and the inactivity time, an activation time necessary for the at least one cold cathode lamp (7) to reach a nominal ultraviolet radiation output;
controlling the power supply of the at least one lamp (7) so as to keep it, even in the absence of an object (2) to be disinfected inside the device (1), at least partially powered in a standby configuration, in order to keep the activation time below a predefined value.

9. The method according to claim 8 and comprising at least the further step of disinfecting an object (2) arranged inside the device (1), in a time in the order of tens of seconds, by removing at least 99.9% of the pathogens; in particular, during the step of disinfecting the object (2), at least one LED auxiliary lighting system (11) arranged at the at least one lamp (7) is switched on so as to reduce the activation time.

10. The method according to claim 8, carried out by a device (1) for disinfecting objects (2) that comprises:
a box-shaped body (3), which determines inside thereof a disinfection chamber (4) and is openable on at least one portion (5) thereof to allow inserting an object (2) to be disinfected inside the disinfection chamber (4);
at least one cold cathode lamp (7), mounted internally the box-shaped body (3) and configured to irradiate the object (2) to be disinfected with an ultraviolet radiation;
one or more detection devices (9) configured to detect environmental conditions inside the disinfection chamber (4);
a control system (10) configured to determine, based on at least the detected environmental conditions and the inactivity time of the device (1), an activation time required for the at least one lamp (7) to reach a nominal ultraviolet radiation output; the control system (10) being further configured to control the power supply of the at least one lamp (7).
